(19) United States
(12) Patent Application Publication
(10) Pub. No.: US 2012/0095087 A1
(43) Pub. Date: Apr. 19, 2012

(54) ENHANCED PRODUCTS BY SUSTAINABLE PROCESSES FOR MEDICINAL USE

(76) Inventor: Keith Hyatt, Anaheim, CA (US)

(21) Appl. No.: 13/090,400

(22) Filed: Apr. 20, 2011

Related U.S. Application Data
(60) Provisional application No. 61/393,785, filed on Oct. 15, 2010.
(60) Provisional application No. 61/393,817, filed on Oct. 15, 2010.

(51) Int. Cl. A61K 31/353; A61P 25/00; A61P 25/04; A61P 21/00; A61K 31/05; A61P 29/00

(52) U.S. Cl. 514/454; 514/733

(57) ABSTRACT

A process for producing a composition with bioactive and/or bioavailable Cannabis-derived cannabinoids known to be effective for CB1 and/or CB2 modulation, and a plurality of indications for patients in need. Using a heat cycle process to combine cannabinoids, including but not limited to THC and CBD with flax seed oil and at least one of the triglycerides therein, an extract is formulated which enables substantially profiled and Cannabinoid ratio-balanced aliquots (“miquots”) to be offered for consideration to patients, including non-psychoactive topically and orally delivered products and systems.

### STATEMENT OF PRIORITY AND CROSS-REFERENCE TO RELATED APPLICATIONS UNDER THE PARIS CONVENTION

The instant filing requesting grant of Letters Patent claims full Paris Convention Priority from U.S. Provisional Patent Applications Ser. Nos. 61/393,785 and 61/393,817 (each filed Oct. 15, 2010) respectively in the name of the present inventor, and each of which is expressly incorporated herein by reference as if fully set forth herein.

### BACKGROUND OF THE INVENTION

i. Field Of The Disclosure

The present invention relates to enhanced products by sustainable processes for medicinal use. In particular, the present invention relates to newly formulated and stabilized products for medicinal use, for example, those offered for consideration under the auspices of Alaska Ballot Measure 8, California Proposition 215, Colorado Amendment 20, District of Columbia Amendment 618-622, Hawaii Senate Bill 862, Maine Ballot Question 2, Michigan Proposal 1, Montana Initiative 1448, Nevada Ballot Question 9, New Jersey Senate Bill 119, New Mexico Senate Bill 523, Oregon Ballot Measure 67, Rhode Island Senate Bill 0710, Vermont Senate Bill 76 HB 645, Washington Initiative 692, and related international, national, territorial, or later enacted statutory or legislative authority.

In order to overcome longstanding needs to provide alternative medicine sources to patients in need of the same, the present inventor has researched both historical and technical solutions to these issues. A detailed review of the literature failed to reveal any disclosure teaching what has been disclosed by the present inventor, in terms of profiling, compounding and delivering objects of the present invention. For this reason, and as set forth and claimed below, the utilities of the present solution, as offered for consideration herein are respectfully proposed to constitute invention, as defined by statute.

From Mesopotamian times until now, healers and shamans have recognized properties of elements derived from the Cannabis plant that are helpful to patients in need. Detailed mechanisms proposed for the same have been set forth below, as derived from the extensive literature surveyed along with historical and technical records. What is now patent is that selected use of the bioactive components of said plant, or cannabinoids, when managed properly, can be safely and effectively deployed medicinally, both with and without psychoactive effects. The present inventor has developed extracts allowing (what are believed to be naturally competing) levels of cannabinoids to be administered by moderating levels of THC (Tetrahydrocannabinol) relative to CBD (cannabidiol) and CBN (cannabinol) levels.

In essence, contrary to prior art collected and studied, the present inventions selectively isolate non-psychoactive cannabinoids so that they can be delivered to, and safely administered to, those in need. By addressing this longstanding need, to bifurcate psychoactive effects from other helpful properties and create medicinal products has not been fully addressed prior to the advent of the instant teachings. Likewise, no literature suggests or motivates those skilled in the art to undertake applicant's unique formulational approach, let alone prior art, therefore making the instant teachings progress in science and the useful arts, it is respectfully proposed, as fully disclosed herein, and claimed below.

By way of example, it is understood and believed that by chemically profiling different strains, phenotypes and expressed genetic variations can be selectively combined, hybridized, or otherwise selected to control the levels of various cannabinoids and their natural or inherent ratios managed, revised or altered as required. Combining strain profiling with user needs' profiling and database management is the interface between compassionate use and personalized medicine. Monitoring just three main cannabinoids; THC, CBD and

CBN, can bridge the phenomenological gaps which exist with respectively different and genetically pre-selected strains, families and groupings. It is finally respectfully proposed that allowing patients to have full access to data about THC/CBD/CBN levels can substantially enhance their ability to make efficacious choices about the correct form and type of medicinal treatment which they choose.

ii. Setting of the Invention

Cannabinoid receptors are a class of cell membrane receptors under the G protein-coupled receptor superfamily. CB1 and CB2 are known cannabinoid receptors. Cannabinoid receptors are activated by three major group of ligands; endocannabinoids (known to be produced by the mammalian body), plant cannabinoids (such as THC, produced by the Cannabis plant) and synthetic cannabinoids (such as HU-210). All of the endocannabinoids and plant cannabinoids are lipophilic, i.e. fat soluble, compounds. Tetrahydrocannabinol (THC), cannabidiol (CBD) and cannabinol (CBN) are the most prevalent natural cannabinoids and have received the most study. Some other common cannabinoids are listed in U.S. Pat. No. 7,179,800, expressly incorporated herein by reference, as if fully set forth herein.

Phytocannabinoids are compounds found in the Cannabis plant that are structurally related to tetrahydrocannabinol (THC). Cannabis plants can exhibit wide variation in the quantity and type of cannabinoids they produce. The mixture of cannabinoids produced by a plant is known as the plant's cannabinoid profile. Selective breeding has been used to control the genetics of plants and modify the cannabinoid profile. Cannabis plants used as fiber (commonly called hemp) are bred for fiber and are known to have low levels of psychoactive chemicals like THC. Cannabis may also be selectively bred for high CBD content. However, prior to the advent of the instant teachings, no formulational approaches have been known to do this.

Plant or Phytocannabinoids, also called natural cannabinoids, herbal cannabinoids, and classical cannabinoids, are only known to occur naturally in significant quantity in the cannabis plant, and are concentrated in a viscous resin that is produced in glandular structures known as trichomes. In addition to cannabinoids, the resin is rich in terpenes, which are largely responsible for the odour of the cannabis plant. (Some cannabinoids are described in U.S. Pat. No. 5,227,537, incorporated by reference expressly, as if fully set forth herein. Other identified cannabinoids are similarly disclosed in Agurell, et al., Pharmacological Review, 38:31-43, 1986, which is also incorporated by reference). Phytocannabinoids are nearly insoluble in water but are soluble in lipids, alcohols, and other non-polar organic solvents. However, as phenols, they form more water-soluble phenolate salts under strongly alkaline conditions. At least 100 cannabinoids have been isolated from the cannabis plant. Tetrahydrocannabinol (THC), cannabidiol (CBD) and cannabinol (CBN) are the most prevalent natural cannabinoids and have received the most study.

Endocannabinoids, found in the nervous and immune systems of animals and that activate cannabinoid receptors, are substances produced from within the body that activate cannabinoid receptors.

Synthetic cannabinoids encompass a variety of distinct chemical classes: the classical cannabinoids structurally related to THC, the non-classical cannabinoids including the aminoalkylindoles, 1,5-diarylpyrazoles, quinolines and arylsulphonamides, as well as eicosanoids related to the endocannabinoids.

It is well known that cannabinoids bind reversibly and stereo-selectively to the cannabinoid receptors. The affinity of an individual cannabinoid to each receptor determines the effect of that cannabinoid. (British Journal of Pharmacology, 2003 March; 138(5): 767-774).

A correlation between inflammation in both the peripheral nervous system and in the central nervous systems (CNS) has been found with respect to some cannabinoid receptors such as CB2. (British Journal of Pharmacology, 2008 Jan; 153(2): 277-85. Epub Oct. 15, 2007).

Cannabinoid receptor agonists, may be useful in the treatment of pain, inflammation and autoimmune diseases. An agonist is a chemical that binds to a receptor of a cell and triggers a response by that cell. Agonists often mimic the action of a naturally occurring substance.

Whereas an agonist causes an action, an antagonist blocks the action of the agonist. A receptor antagonist is a type of receptor ligand, or drug that does not provoke a biological response itself upon binding to a receptor, but blocks or dampens agonist-mediated responses. In pharmacology, antagonists have affinity but no efficacy for their cognate receptors, and binding will disrupt the interaction and inhibit the function of an agonist or inverse agonist at receptors. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist—receptor complex, which, in turn, depends on the nature of antagonist receptor binding. The majority of drug antagonists achieve their potency by competing with endogenous ligands or substrates at structurally-defined binding sites on receptors.

All-natural cannabinoids are derived from their respective 2-carboxylic acids (2-COON) by decarboxylation (catalyzed by heat, light, or alkaline conditions).

Tetrahydrocannabinol (THC) is the primary psychoactive component of the plant. It has been shown to ease moderate pain (analgesic) and to be neuroprotective. THC has approximately equal affinity for the CB1 and CB2 receptors. Although years of selective breeding have sought to maximize THC levels, it is readily possible to down-modulate THC levels, as may be needed for many patients. However, no prior art teachings were developed which use flaxseed oil and Cannabis material, or ganja.

Ganja is the historical term which refers to the sum total of all plant parts. Sustainable agriculture involves making use of all offered for consideration. Not unlike the Native Americans' approach to preserving and harvesting each precious morsel in a sacrificed bison, the process of the present invention wastes no remnants.

Cannabidiol (CBD) is a cannabinoid found in Cannabis. It is a major constituent of the plant, representing up to 40% in its extracts. [Grlic, L. (1976). “A Comparative Study on Some Chemical and Biological Characteristics of Various Samples of Cannabis Resin.” U.N. Bulletin on Narcotics 14: 37-46].

It has displayed sedative effects in animal tests [Pickens, J. (1981). “Sedative Activity of Cannabis in Relation to its Delta′-trans-tetrahydrocannabinol and Cannabidiol content.” British Journal of Pharmacology, 72 (4): 649-56]. Some research, however, indicates that CBD can increase alertness [Nicholson, A.; C. Turner, B. Stone, and P. Robson (June 2004). “Effect of Delta-9-tetrahydrocannabinol and Cannabidiol on Nocturnal Sleep and Early-morning Behavior in Young Adults,” Journal of Clinical Psychopharmacology 24 (3): 305-13.]. It may decrease the rate of THC clearance from the body, perhaps by interfering with the metabolism of THC in the liver. Cannabis indica strains often feature higher CBD content.

Medically, it has been shown to relieve convulsion, inflammation, anxiety, and nausea, as well as inhibit cancer cell growth [Mechoulam, R.; M. Peters, Murillo-Rodriguez (21 Aug 2007). “Cannabidiol-Recent Advances,” Chemistry & Biodiversity 4 (8): 1678-1692.] Recent studies have shown cannabidiol to be as effective as atypical antipsychotics in treating schizophrenia[6]. [Zuardi, A.; J. A. S. Crippa, J. E. C. Hallak, F. A. Moreira, F. S. Guimarães (2006). “Cannabidiol, a Cannabis sativa Constituent, as an Antipsychotic Drug,” Brazilian Journal of Medical & Biological Research, 39 (4): 421-429.] Studies have also shown that it may relieve symptoms of dystonia [Consroe, P; Sandyk, R; Snider, SR (1986). “Open Label Evaluation of Cannabidiol in Dystonic Movement Disorders,” International Journal of Neuroscience, 30 (4): 277-82; Snider, Stuart R. and Consroe, Paul. (1985). “Beneficial and Adverse Effects of Cannabidiol in a Parkinson Patient with Sinemet-Induced Dystonic Dyskinesia”. Neurology, (Suppl. 1) p. 201. ].

Cannabidiol has no affinity for CB1 and CB2 receptors but acts as an indirect antagonist of cannabinoid agonists.[Mechoulam, R.; M. Peters, Murillo-Rodriguez (21, Aug. 2007). “Cannabidiol-Recent Advances,” Chemistry & Biodiversity 4 (8): 1678-1692]. Recently it was found to be an antagonist at the putative new cannabinoid receptor, GPR55, a GPCR expressed in the caudate nucleus and putamen [Ryberg E, Larsson N, Sjögren S, et al. (2007). “The Orphan Receptor GPR55 is a Novel Cannabinoid Receptor,” British Journal of Pharmacology 152 (7): 1092.] Cannabidiol has also been shown to act as a 5-HT1A receptor agonist, [Russo EB, Burnett A, Hall B, Parker K K (August 2005). “Agonistic properties of cannabidiol at 5-HT1a receptors,” Neurochemical Research 30 (8): 1037-43.] an action which is involved in its antidepressant [Zanelati T, Biojone C, Moreira F, Guimarães F, Joca S (December 2009). “Antidepressant-like Effects of Cannabidiol in Mice: Possible Involvement of 5-HT Receptors,” British Journal of Pharmacology 159 (1): 122-8] and [Resstel LB, Tavares R F, Lisboa S F, Joca S R, Correa F M, Guimarães F S (January 2009). “5-HT1A Receptors are Involved in the Cannabidiol-induced Attenuation of Behavioral and Cardiovascular Responses to Acute Restraint Stress in Rats,” British Journal of Pharmacology 156 (1): 181-8.], anxiolytic [Campos AC, Guimaraes FS (August 2008). “Involvement of 5HT1A Receptors in the Anxiolytic-like effects of Cannabidiol Injected into the Dorsolateral Periaqueductal Gray of Rats,” Psychopharmacology 199 (2): 223-30.] and neuroprotective effects [Mishima K, Hayakawa K, Abe K, et al. (May 2005). “Cannabidiol Prevents Cerebral Infarction via a Serotonergic 5-hydroxytryptamine1A Receptor-dependent Mechanism,” Stroke: A Journal of Cerebral Circulation 36 5): 1077-82] and [Hayakawa K, Mishima K, Nozako M, et al. (March 2007). “Repeated treatment with cannabidiol but not Delta9-tetrahydrocannabinol has a neuroprotective effect without the development of tolerance,” Neuropharmacology 52 (4): 1079-87.]

Cannabidiol has also been shown to inhibit cancer cell growth with low potency in non-cancer cells. Although the inhibitory mechanism is not yet fully understood, Ligresti et al. suggest that “cannabidiol exerts its effects on these cells through a combination of mechanisms that include either direct or indirect activation of CB2 and TRPV1 receptors, and induction of oxidative stress, all contributing to induce apoptosis.”[Ligresti A., Moriello A., Starowicz K., et al. (2006). “Antitumor Activity of Plant Cannabinoids with Emphasis on the Effect of Cannabidiol on Human Breast Carcinoma,” Journal of Pharmacology & Experimental Therapeutics 318 (3): 1375-87.] It has also been reported that CBD shows promise for controlling the spread of metastatic breast cancer. In vitro CBD downregulates the activity of the gene ID1 which is responsible for tumor metastasis [McAllister S., Christian R., Horowitz M., Garcia A., Desprez P. (2007). “Cannabidiol as a Novel Inhibitor of Id-1 Gene Expression in Aggressive Breast Cancer Cells,” Molecular Cancer Therapeutics 6 (11): 2921-7.]

Cannabinoids are traditionally separated from the plant by extraction with organic solvents. Hydrocarbons and alcohols are often used as solvents. However, these solvents are flammable and many are toxic. Supercritical solvent extraction with carbon dioxide is an alternative technique. Although this process requires high pressures (73 atmospheres or more), there is minimal risk of fire or toxicity, solvent removal is simple and efficient, and extract quality can be well-controlled. Once extracted, cannabinoid blends can be separated into individual components using wiped film vacuum distillation or other distillation techniques. However, to produce high purity cannabinoids, chemical synthesis or semisynthesis is generally required.

Medications containing natural or synthetic cannabinoids or cannabinoid analogs are:
Dronabinol (Marinol), is ≢9-tetrahydrocannabinol (THC), used as an appetite stimulant, anti-emetic, and analgesic
Nabilone (Cesamet), a synthetic cannabinoid and an analog of Marinol. It is Schedule II unlike Marinol, which is Schedule III
Sativex, a cannabinoid extract oral spray containing THC, CBD, and other cannabinoids used for neuropathic pain and spasticity in 22 countries including England, Canada and Spain. Sativex develops whole-plant cannabinoid medicines.
Rimonabant (SR141716), a selective cannabinoid (CB1) receptor antagonist used as an anti-obesity drug under the proprietary name Acomplia. It is also used for smoking cessation.

Other synthetic cannabinoids include:
JWH-018, a potent synthetic THC analogue discovered by Dr. John W. Huffman at Clemson University. It is being increasingly sold in legal smoke blends collectively known as “spice”. Several countries and states have moved to ban it legally
CP-55940, produced in 1974, this synthetic cannabinoid receptor agonist is many times more potent than THC
Dimethylheptylpyran
HU-210, about 100 times as potent as THC[28]
HU-331 a potential anti-cancer drug derived from cannabidiol that specifically inhibits topoisomerase II
SR144528, a CB2 receptor antagonists
WIN 55,212-2, a potent cannabinoid receptor agonist
JWH-133, a potent selective CB2 receptor agonist
Levonantradol (Nantrodolum), an anti-emetic and analgesic but not currently in use in medicine

### FIGURES

FIG. 1 is a block diagram showing process steps to an exemplary implementation of an F-C extract, according to the teachings of the present invention.

### SUMMARY

According to embodiments, there is provided a process for producing a cannabinoid and triglyceride composition; the process comprising, in combination; adding at least about one gallon of cold pressed flax seed oil having with a 214 F burn point to a container which can be covered; adding at least about 228 grams of ground up plant materials per about one gallon from material derived from Cannabis plants; covering for between about 8 and up to 12 hours the container and cycling back and forth between a maximum heat of less that 214 degrees F. and a minimum heat of between about 195-198 F while mechanically agitating while alternating between maximum and minimum heat; cool a resulting admixture down to room temperature; and, separating the plant material from the liquid wherein the liquid forms an F-C extract.

According to embodiments, there is provided a method for administering medicinal marijuana to mammals, comprising, in combination; profiling Cannabinoid levels in Cannabis plant material, extracting a pharmaceutically effective dose, and combining the same with a carrier.

Broadly stated, the instant teachings provide a process for producing a composition with bioactive and/or bioavailable CB1 and/or CB2 modulation. Using a heat cycle process to combine cannabinoids, including but not limited to THC and CBD with flax seed oil and at least one of the triglycerides therein, an extract is formulated which enables substantially profiled and Cannabinoid ratio-balanced aliquots to be offered for consideration to patients, including non-psychoactive topically and orally delivered products and systems.

i. DETAILED DISCLOSURES

Various exemplary implementations of the present invention share and are based upon the principle that the combination of a cannabinoid containing composition and flax oil linseed oil, also known as flax seed oil, is a clear to yellowish oil obtained from the dried ripe seeds of the flax plant (Linum usitatissimum, Linaceae). The oil is obtained by cold pressing, sometimes followed by solvent extraction. Linseed oil is a mixture of various triglycerides that differ in terms of their fatty acid constituents. For linseed oil, the constituent fatty acids are of the following types:

The saturated acids palmitic acid (about 7%) and stearic acid (3.4-4.6%),

The monounsaturated oleic acid (18.5-22.6%),

The doubly unsaturated linoleic acid (14.2-17%),

The triply unsaturated omega-3 fatty acid a-linolenic acid (51.9-55.2%).

Food-grade flax seed oil is cold-pressed, obtained without solvent extraction, and marketed as edible flax seed oil. Fresh, refrigerated and unprocessed, linseed oil is used as a nutritional supplement. It contains the highest level of omega-3 fatty acids among vegetable oils,[3] especially alpha-linolenic acid, which may be beneficial for reducing inflammation leading to atherosclerosis. Diane H. Morris. “Flax Reduces Inflammation Leading to Atherosclerosis” (PDF). New Flax Facts. Flax Council of Canada. Flax Council of Canada, 465-167 Lombard Ave., Winnipeg, MB, Canada R3B OT6, www.flaxcouncil.ca. The extracted linseed oil does not contain the ligand found in flax seed, “Flax-A Healthy Food”. Flax Council of Canada. Retrieved 2008, Jan. 24.

ii. DEFINITIONS OF THE DISCLOSURE

A composition of triglycerides from flax seed oil and cannabinoids derived from the cannabis plant is hereinafter also referred to as an F-C extract which is at least one of bioactive and bioavailable with respect to at least one of CB1 and CB2 receptors. In some instances at least one of said cannabinoids is CBD or THC.

A miquot for the purpose of the present invention is a profiled dose allocation of a particular form or embodiment, as customized or quasi-customized to a patient's profiled user needs map.

A pharmaceutically effective dose is defined by that threshold of efficacy by which a patient in need of treatment can receive a benefit.

Those of ordinary skill in the art will recognize that to effect a cannabinoid receptor and be bioactive/bioavailable such effect may include up regulation or down regulation of the receptor via modulation of other pathways or products as well as direct receptor interactions.

The stability and low toxicity of the F-C extract makes it useful as a liquid additive for consumption and/or topical use.

The F-C extract may be combined with dietary items to be consumed as a dietary supplements, beverages, food or tonic. In some instances, the amount of F-C extract added per use is F-C extract into aliquots of at least about between 0.05 and 0.25 ml of volume.

The F-C extract may also be combined with pharmaceutically acceptable carriers to be applied as a lotion, salve, cream, rinse, soap, shampoo, conditioner, cosmetic, and analgesic. In some instances, the amount of F-C extract added per use is F-C extract into miqouts of at least about between 0.05 and 0.25 ml of volume.

The F-C extract may also treat medical conditions such as inflammation, tension, loss of appetite, nervousness, and pain. In some instances, the amount of F-C extract added per use is F-C extract into doses of at least about between 0.05 and 0.25 ml of volume.

The F-C extract may also be combined with pharmaceutically acceptable carriers to form medicines for oral, inhaled, topical, injected, ingested or suppository use. In some instances, the amount of F-C extract added per use is F-C extract into miqouts of at least about between 0.05 and 0.25 ml of volume.

At room temperatures the F-C extract should not rancidify for about 2 to about 60 days. At about 36 to 42 degrees F. the F-C extract should not rancidify for about 30 to about 200 days. At about 20 to 30 degrees F. the F-C extract should not rancidify for about 30 to about 200 days.

In at least one exemplary implementation one or more cannabinoids is combined with flax seed oil resulting in a composition containing at least one of a bioactive and bioavailable Cannabinoid 2 (CB2) receptor agonist and triglycerides.

In at least one exemplary implementation one or more cannabinoids are combined with flax seed oil resulting in a composition containing at least one of a bioactive and bioavailable Cannabinoid 2 (CB2) receptor antagonist and triglycerides.

In at least one exemplary implementation one or more cannabinoids are combined with flax seed oil resulting in a composition containing at least one of a bioactive and bioavailable Cannabinoid 1 (CB1) receptor agonist and triglycerides.

In at least one exemplary implementation one or more cannabinoids are combined with flax seed oil resulting in a composition containing at least one of a bioactive and bioavailable Cannabinoid 1 (CB1) receptor antagonist and triglycerides.

In at least one exemplary implementation one or more cannabinoids are combined with flax seed oil resulting in a composition containing at least a bioactive/bioavailable Cannabinoid 1 (CB1) receptor agonist or antagonist and a bioactive/bioavailable Cannabinoid 2 (CB2) receptor agonist or antagonist and triglycerides.

In at least one exemplary implementation one or more cannabinoids are combined with flax seed oil resulting in a composition containing a cannabinoid receptor agonist or antagonist and triglycerides.

In at least one exemplary implementation one or more cannabinoids are combined with flax seed oil the resulting composition attaching at least one of a Cannabinoid 2 (CB2) receptor agonist or antagonist and Cannabinoid 1 (CB1) receptor agonist or antagonist to the carbon chain of a triglyceride from a flax seed oil.

In at least one exemplary implementation one or more cannabinoids are combined with flax seed oil and a pharmaceutically accepted carrier, included but not limited to lotions, oils and creams, resulting in a composition containing a bioactive Cannabinoid receptor agonist or antagonist and triglyceride that is absorbed by the skin.

In at least one exemplary implementation one or more cannabinoids are combined with flax seed oil and a pharmaceutically accepted carrier, included but not limited to lotions, oils and creams, resulting in a composition containing a bioactive Cannabinoid receptor agonist or antagonist and triglyceride that may be absorbed by mammalian systems.

In at least one exemplary implementation one or more cannabinoids are combined with flax seed oil and a digestible carrier forming a beverage, the carrier including but not limited to at least one of diary, fruit, yogurt, coffee, tea, water, vegetable, grains, alcohol, distilled spirits and containing a bioactive/bioavailable Cannabinoid receptor agonist or antagonist wherein at least a portion of the Cannabinoid receptor agonists or antagonists pass through the stomach and is absorbed in the gut.

In order to better control food quality and compatibility with respective miquots of F-C extract, batches are developed which are drug-free.

In at least one exemplary implementation one or more cannabinoids are combined with flax seed oil and a digestible carrier forming a food , the food including but not limited to at least one of grain, meat, poultry, fish, fruit, vegetable and containing a bioactive/bioavailable Cannabinoid receptor agonist or antagonist wherein at least a portion of the Cannabinoid receptor agonists or antagonists pass through the stomach and is absorbed in the gut.

In at least one exemplary implementation one or more cannabinoids are combined with flax seed oil forming a substantially stable liquid additive containing a bioactive or bioavailable Cannabinoid receptor agonist or antagonist bound to a triglyceride. Stability at room temperature (non-refrigerated), of the liquid additive, is between about 24 hours to about 30 days and is dependant on the exposure to higher temperatures. In some instances the stability at room temperature is between about 24 hours to about 10 days. In some instances the stability at room temperature is between about 12 hours to about 5 days. In some instances the stability of the liquid additive, when refrigerated is between about 24 hours and about 90 days.

It should be appreciated that for simplicity and clarity of illustration, elements shown in the Figure(s) have not necessarily been drawn to scale. For example, the dimensions of some of the elements are exaggerated relative to each other for clarity. Further, where considered appropriate, reference numerals have been repeated among the Figure(s) to indicate corresponding elements.

### Example 1

Step 1: Add about one gallon of flax seed oil cold pressed preferably kosher with a 214 degrees F. burn point to a container.

Step 2: Add about 228 grams of plant materials from parts of a Cannabis plant. The material should be ground up to a course particulate. The mix need not be homogenous in particulate size.

Optional Step 3: Place particulate in a divider material (such a cheese cloth) that contains the particulate and allows flow of oil into the divider and oil and cannabinoids out of the divider material.

Step 3: Place particulate in the flax oil containing container.

Over 8-12 hours cycle back and forth between heat max of 213 degrees F. and cool down to between 195-198 degrees F. as follows:

Step 4: Heat the covered container with flax oil and cannabis particulate to a maximum temperature of less than 214 degrees F. and most preferably between about 213 degrees F. and 214 degrees F. and hold temperature for about 90 to 120 minutes.

Step 5: Agitate the contents of said container for about 30 seconds to about 100 seconds.

Step 6: Lower the temperature of said container to range of 195-198 degrees F. for about 90 to 120 minutes.

Step 7: Repeat steps 4 through 6 for a time period between about 5 and 20 hours. Most preferably between 8 and 12 hours.

Step 8: Cool down said container and separate the flax oil with cannabinoids also known as the “F-C extract”.

### Example 2

Step 1: Add about one gallon of flax seed oil cold pressed preferably kosher with a 214 degrees F. burn point to a container.

Step 2: Add about 228 grams of plant materials from parts of a Cannabis plant. The material should be ground up to a course particulate. The mix need not be homogenous in particulate size.

Optional Step 3: Place particulate in a divider material (such as cheese cloth) that contains the particulate and allows flow of oil into the divider and oil and cannabinoids out of the divider material.

Step 3: Place particulate in the flax oil containing container.

Over 8-12 hours cycle back and forth between heat max of 213 degrees F. and cool down to between 195-198 degrees F. as follows:

Step 4: Heat the covered container with flax oil and cannabis particulate to a maximum temperature of less than 214 degrees F. and most preferably between about 213 degrees F. and 214 degrees F. and hold temperature for about 90 to 120 minutes.

Step 5: Agitate the contents of said container for about 30 seconds to about 100 seconds.

Step 6: Lower the temperature of said container to range of 195-198 degrees F. for about 90 to 120 minutes.

Step 7: Repeat steps 4 through 6 for a time period between about 5 and 20 hours. Most preferably between 8 and 12 hours.

Step 8: Cool down said container and separate the flax oil with cannabinoids also known as the “F-C extract”.

Step 9: Add at least one of oil of rosemary and lemon oil to the “F-C extract”.

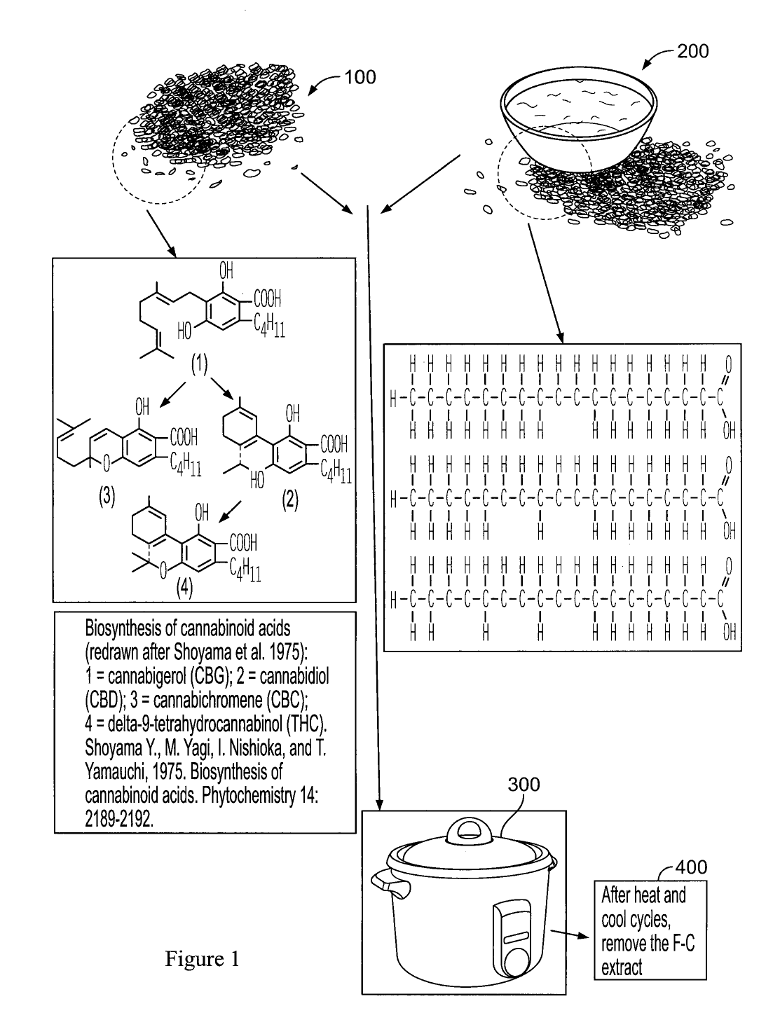

The F-C extract (see generally FIG. 1) formed in a containment vessel such as a crock pot or pressurized vessel as described in examples 1 and 2. The container with flax oil and cannabis particulate is temperature controlled. All temperatures and times listed in the process examples 1 and 2 are based on a pressure of near atmospheric. Increasing the pressure on the flax seed oil and cannabis mixture can be used to reduce cycle times between the higher and lower temperatures. Those of ordinary skill in the art will also recognize that pressure increase may also reduce the necessary temperatures to stress (high temperature) the triglycerides (flax seed oil carbon chains) and distress (lower temperature) the triglycerides carbon chains.

It is believed that in some instances the periods of stress, at a temperature that is below the threshold temperature for degradation of the triglycerides, reversibly alters the conformational shape of the carbon chain thereby promoting attachment of one or more cannabinoids thereto.

In some exemplary implementations the F-C extract contains cannabinoids bound to a triglyceride derived from the flax seed oil. In some instances the cannabinoids bound are at least one of a CB1 and CB2 receptor modulator. In some instances, the F-C extract is suitable for ingestion and ingestion is via a liquid with a carrier that may include fruit juices, vegetable juices, water, cream, milk, yogurt, wine, distilled sprits, beer, coffee, tea, herbal teas, and carbonation.

In some exemplary implementations the F-C may be combined with a pharmaceutically acceptable carrier including but not limited to sodium cromoglycate, bronchodilators and glucocorticosteroids such as those used in pressurized metered-dose inhaler (pMDI) for inhalation. Said F-C extract may also be delivered for inhalation via wet nebulizers may be subdivided into jet and ultrasonic models (See, generally CMAJ•NOV. 30, 1999; 161 (11 Suppl) S1 © 1999 Canadian Medical Association or its licensors).

### Example 3

Combining the F-C extract with topical carriers is embodied in multiple forms, and has achieved current medical success. Both a massage oil and a pain-mitigating cream have been developed and made available to properly credentialed users under compassionate use legislation. (See, for example, the GJ® brand of rub and massage oil available from Premium Organic Treatments, Anaheim, Calif. 92805.) Likewise, a lip balm has been developed and each of these proprietary formulations, as explained herein, and claimed below leverage the proprietary extracting system which is driven by the Cannabinoid profiling which is done by independent and highly credible testing laboratories.

Likewise, the GANJA DERM line of sustainable cosmetics was evolved to generate consumer knowledge of an interest in the base cosmetological, supplement-based, organic, vegan and related brand identities, for cosmetics.

(Available from OMG Outkast Marketing entities of Orange County, Calif., 92677.)

Essentially, as further developed throughout this document and claimed hereafter, the sustainable nature of the instant teachings drives use of the entire plant for purposes of generating extracts. Per examples one and two above, an F-C extract is readily combinable with proprietary compounds, mixtures, admixtures and related collections of desired ingredients. Those skilled in the art readily understand how to compound, entabulate, mix, and/or otherwise combine the instant teachings with a plurality of vehicles for delivery to humans and/or other mammals in need of the same.

Similarly, Cannabinoid profiling enables formulators to array and establish preferred ratios for THC, CBD and CBN, inter alia. By having numerous mechanisms to store and track such data, those skilled in the art can and do have the ability to formulate various products, derived from this sustainable base for us by at least two groups having needs for medicines based upon cannabis—namely, those who desire to have psychoactive effects and those who do not. It is further respectfully proposed that among the proprietary aspects of the instant teachings are psychoactive-free formulations which remain safe and effective and modulate pain, inflammation, muscle issues, insomnia and the multiplicity of other ailments those having needs for the products of the present invention seek.

### Example 4

F-C extracts as proposed above were combined with ingestible carriers, namely fruit juice based smoothies having desired levels of natural fruit, sugars and fats (for example). Patients needing anti-inflammatory, pain-modulating and anti-distonic support were provided input on construction of appropriate miquots, namely unit-dose administration suggestions which addressed their enumerated health concerns. Smoothies have base-flavors, stemming from Vaccinum vites idea (blueberry) or any major fruit group were proposed and provided in transportable unit-dose suspensions. Patients' data on usage history, preferred and desired medically prognosed response were stored in a database and Cannabinoid profiles for various F-C extracts reviewed and optimized products selected. Chronically afflicted sufferers of amytrophic lateral sclerosis, multiple sclerosis, and advanced neuropathic degeneration (from Diabetes mellitus and Parkinson's-like disease etiologies) reported no adverse events and returned for multiple subsequent visits.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the invention both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementations, a method or process implementations, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular exemplary implementations, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative exemplary implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

What is claimed is:

1. A process for producing a cannabinoid and triglyceride composition, the process comprising, in combination:
adding at least about one gallon of cold pressed flax seed oil having with a 214 degrees F. burn point to a container which can be covered;
adding at least about 228 grams of ground up plant materials per about one gallon from material derived from Cannabis plants;
covering for between about 8 and up to 12 hours the container and cycling back and forth between a maximum heat of less that 214 degrees F. and a minimum heat of between at least about 195-198 degrees F. while mechanically agitating and alternating between maximum and minimum heat;
cool a resulting admixture down to room temperature; and, separating the plant material from the liquid wherein the liquid forms an F-C extract.

2. The process of claim 1, further comprising:
the cycle times being between 90 and 120 minutes for each maximum heat and each minimum heat cycle; and, the duration of the mechanical agitation being between at least about 30 and approximately 148 seconds.

3. The process of claim 2, further comprising the step of:
placing said plant material within a filtering divider material which both contains the plant material and reduces the flow of plant materials out of the filtering divider material.

4. The process of claim 3, wherein said cannabinoids are at least one of CBD and THC.

5. A product by the process of claim 4, further comprising:
Aliquots of the F-C extract being separated into single doses by volume, profile, and intended use;
Each of said aliquots being conformed into miquots for further processing purposed.

6. The product of claim 5, wherein said single dose ranges from at least about 0.05 to about 0.25 ml of volume.

7. The product of claim 6, further comprising combining said F-C extract with an ingestible carrier.

8. The product of claim 6, further comprising combining said F-C extract with a topical carrier.

9. The product of claim 6, further comprising combining said F-C extract with an inhaled topical carrier.

10. A product, according to claim 8, wherein said topical carrier further comprises, in combination:
Extra Virgin olive oil;
High Oleic Sunflower Oil;
Coconut Oil; and
Vitamin E.

11. The product of claim 10, wherein the ratio of CBD to THC precludes psychoactive effects.

12. A product, according to claim 8, wherein said topical carrier further comprises, in combination:
Water;
Goat's Milk;
Hellanthus Annus Oil;
Soya Glycine;
Theobroma Cacao Seed Butter;
Butyospernum;
Park Fruit;
Vegetable Glycerin;
Glycol Copolymer;
Isopropyl Myristate;
Monostearate;
Acrylamide;
Octyldodecanol;
Butylcarbamate;
Palmitate PEG-320;
Idopropanol;
Polysorbate;
Ceterate-14;
Aloe Vera Extract;
Glyceril Stearate;
Carbopol; and
Essential Oils, as determined to maintain consistency, shelf-life, and desiderata enumerated in terms of viscosity.

13. The products of claim 12, wherein the ratio of CBD to THC precludes psychoactive effects.

14. The product of claim 7, said ingestible carrier further comprising a blended fruit-based drink.

15. The product of claim 9, said ingestible carrier further comprising at least one of a crystalline structure and an appropriate chemical nature to be suspended in an aqueous medium.

16. The product of claim 7, said ingestible carrier further comprising a confectionary product selected from the group consisting of brownies, cookies, muffins, Rice-Crispies® brand of snacks and candies.

17. The product of claim 7, said ingestible carrier further comprising at least one of tea, coffee, salad dressing and butter-related products.

18. The product of claim 8, further comprising soaps, shampoos, lip balms and products used as toiletries.

19. The products of claim 18, wherein the ratio of CBD to THC precludes psychoactive effects.

20. A method for administering medicinal marijuana to mammals, comprising, in combination:
Profiling Cannabinoid levels in Cannabis plant material;
Extracting a pharmaceutically effective dose; and
Combining the same with a carrier.
